(19) United States
(12) Patent Application Publication
(10) Pub. No.: US 2006/0019934 A1
(43) Pub. Date: Jan. 26, 2006

(54) Anti-HIV-1 activity of betulinol derivatives

(76) Inventors: Brij B. Saxena, Englewood, NJ (US); Premila Rathnam, Englewood Cliffs, NJ (US); Arkadiy Bomshteyn, Brooklyn, NY (US)

Correspondence Address: Michael L. Goldman;Nixon Peabody LLP, Clinton Square, P.O. Box 31051, Rochester, NY, 14603-1051, US

(21) Appl. No.: 11/131,851

(22) Filed: May 18, 2005

Related U.S. Application Data
(60) Provisional application No. 60/572,812, filed on May 20, 2004.

(51) Int. Cl. A61K 31/56

(52) U.S. Cl. 514/169

(57) ABSTRACT

The present invention relates to a method of inhibiting HIV-1 activity in a cell. This method involves providing a cell infected with HIV-1 and contacting the cell with a compound of Formula I 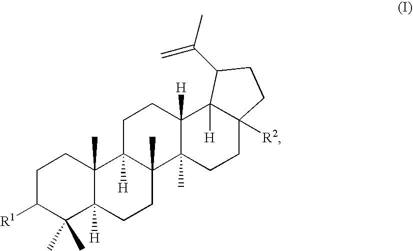 where
R¹ is —CH₃, ═O, —OH, —OCH₃, or —OC(O)CH₃, and
R² is —H, —CH₃, —CHO, —CH₂OH, —CH₂OCH₃, —CH₂OC(O)CH₃, —COCH₃, or —COOH, or a pharmaceutically acceptable salt or derivative thereof, under conditions effective to inhibit HIV-1 activity in the cell. A method of treating HIV-1 infection in a subject is also disclosed. This method involves administering a therapeutically effective amount of a compound of Formula I, or a pharmaceutically acceptable salt or derivative thereof, under conditions effective to treat the subject for HIV-1 infection.

This application claims the priority benefit of U.S. Provisional Patent Application Ser. No. 60/572,812, filed May 20, 2004, which is hereby incorporated by reference in its entirety.

### FIELD OF THE INVENTION

The present invention relates generally to betulinol derivatives and, in particular, to methods of inhibiting HIV-1 activity in a cell and treating HIV-1 infection in a subject.

### BACKGROUND OF THE INVENTION

Human Immunodeficiency Virus (“HIV”), the virus that causes AIDS, has reached pandemic proportions in the world. Some one million people are infected with HIV in the U.S. alone, and more than forty million worldwide. Each day, approximately 12,000 adults and 1,800 children become infected. Currently, there are three classes of drug treatments for HIV, namely, reverse transcriptase (“RT”) inhibitors, such as AZT (3′-azido-3′-deoxythymidine), protease inhibitors, and fusion inhibitors. Common HIV drug therapy includes a cocktail drug regiment, which may utilize, for example, nucleoside analogs like AZT, 2′,3′-dideoxyinosine, and 2′,3′-dideoxycytidine. These drugs act through the inhibition of the HIV reverse transcriptase activity and/or by a mechanism of oligonucleotide chain termination.

However, these currently acceptable treatment drugs are limited by either their toxicity or the emergence of drug-resistant HIV strains (Evers et al., J. Med. Chem. 39:1056-1063 (1996)). In addition, these drugs are costly, difficult to manufacture, and have adverse side effects. Subjects also frequently develop resistance to these drugs. Therefore, the search for new types of anti-HIV compounds is timely and important.

Betulin, or betulinol, is one of the more plentiful triterpenes, constituting up to twenty-four percent of the outer bark of the white birch (Betula alba) and as much as thirty-five percent of the outer bark and about five percent of the inner bark of the Manchurian white birch (Betula platyphylla) (Hirota et al., J.S.C.I. Japan 47:922 (1944)). Betulin also occurs in a free state in the bark of yellow and black birch (Steiner, Mikrochemie, Molisch-Festschrift, p. 405 (1936)), Corylus avellana and Carpinus betulus (Feinberg et al., Monatsh 44:261 (1924); Brunner et al., Monatsh 63:368 (1934); Brunner et al., Monatsh 64:21 (1934)), and Lophopetalum toxicum (Dieterle et al., Arch. Pharm. 271:264 (1933)). The exudate from the bark of Trochodendron aralioides, which constitutes Japanese bird-lime, contains betulin palmitate (Shishido et al., J.S.C.I. Japan 45:436 (1942)). Betulin has also been isolated from rosehips (Zimmermann, Helv. Chim. Acta 27:332 (1944)) and from the seeds of Zizyphus vulgaris Lamarck var. spinosus Bunge (Rhamnaceae) (Kawaguti et al., J. Pharm. Soc. Japan 60:343 (1940)). Ruhemann et al., Brennstoff-Ch. 13:341 (1932) discloses the presence of betulin, allobetulin, and an “oxyallobetulin” in the saponifiable portion of a benzene-alcohol extract of mid-German brown coal. In addition, the following group of lupon-row derivatives from the birch cortex extract have been identified: (a) betulinol, (b) betulinic acid, (c) betulin aldehyde, (d) betulonic acid, and (e) betulone aldehyde (Rimpler et al., Arch. Pharm. Und. Ber. Dtsh. Ppharmaz Jes. 299:422-428 (1995); Lindgren et al., Acta Chem. 20:720 (1966); and Jaaskelainen, P. Papperi Ja Puu-Papper Och Tra. 63:599-603 (1989)).

Birch tree cortex-extracted betulinol was first mentioned as an antiseptic in 1899. Subsequently, compounds singled out from extracts of Hyptis emory and Alnus oregonu, identified as pentacyclic styrenes and their derivatives, were shown to inhibit carcinosarcoma growth (Sheth et al., J. Pharm. Sci. 61:1819 (1972); Sheth et al., J. Pharm. Sci. 62:139-140 (1973)). It has been suggested that betulinic acid is the main anti-tumor agent in the mixture of terpenoids (Tomas et al., Planta Medicina 54:266-267 (1988); Ahmat et al., J. Indian Chem. Soc. 61:92-93 (1964)). In particular, betulinic acid showed cytotoxic activity against carcinoma cell line CO-115 of the large intestine (LD 50=0.375 mg/ml) (Ukkonen et al., Birch Bark Extractive Kemia Kemi 6:217 (1979)). U.S. Patent Application Publication No. 2003/0036540 to Bomshteyn et al., discloses betulinol derivatives and betulinol-antibody conjugates useful in treating cancer.

Betulinol (lup-20(29)-ene-3.beta., 28-diol) is commercially available (e.g., Sigma Chemical Co., St. Louis, Mo.) and is described for example, in “Merck 1212,” The Merck Index, 11 th ed. (1989), and Simonsen et al., The Terpenes Vol. IV, Cambridge U. Press, pp. 187-328 (1957).

The chemical structure of betulinol is:

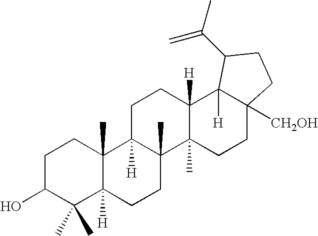

Betulinol has been shown to have anti-viral activity, including anti-herpes virus activity (U.S. Pat. No. 5,750,578 to Carlson et al.) and anti-HIV activity (U.S. Pat. No. 6,172,110 to Lee et al.; Sun et al., J. Med. Chem. 41:4648-4657 (1998)). Certain betulinol derivatives have also been investigated with regard to potential for anti-viral activity.

Betulonic acid and derivatives thereof (Hashimoto et al., Bioorg. Med. Chem. 5:2133-2143 (1997); Sun et al., J. Med. Chem. 41:4648-4657 (1998)), betulinic acid and derivatives thereof, dihydrobetulinic acid and derivatives thereof (Hashimoto et al., Bioorg. Med. Chem. 5:2133-2143 (1997); Sun et al., J. Med. Chem. 45:4271-4275 (2002); Kashiwada et al., Bioorg. Med. Chem. Lett. 11:183-185 (2001); Kashiwada et al., J. Med. Chem. 39:1016-1017 (1996); Flekhter et al., Bioorg. Khim. 28:543-550 (2003); “Betulinic Acid Derivatives in AIDS,” Marketletter (May 2, 1994); DeClercq, Med. Res. Rev. 20:323-349 (2000); Vlietinck et al., Plant Med. 64:97-109 (1998); Soler et al., J. Med. Chem. 39:1069-1083 (1996); Evers et al., J. Med. Chem. 39:1056-1068 (1996); U.S. Pat. No. 5,468,888 to Bouboutou et al., U.S. Pat. No. 5,697,828 to Lee et al., U.S. Pat. Nos. 5,869,535, 6,225,353, 6,495,600, and 6,569,842 to Pezzuto, U.S. Pat. No. 6,048,847 to Ramadoss et al., and U.S. Pat. No. 6,403,816 to Jaggi et al.; and PCT Application Publication No. WO 96/39033 to Lee et al.), betulin diacetate (Sun et al., Med. Chem. 41:4648-4657 (1998)), and betulone aldehyde (U.S. Pat. Nos. 5,869,535, 6,225,353 and 6,495,600 to Pezzuto et al.) have been investigated with regard to potential for anti-HIV activity. In addition, certain betulin derivatives, including betulin diacetate (U.S. Pat. No. 5,750,578 to Carlson) and betulinic acid (U.S. Pat. No. 6,214,350 to Hwang) have been shown to exhibit anti-herpes virus activity.

Unfortunately, however, many of these betulinol derivative compounds have significant drawbacks to their use. Betulin diacetate and betulonic acid, for example, have been shown to exhibit a low therapeutic index (Sun et al., J. Med. Chem. 41:4648-4657 (1998)). In addition, certain betulinic acid derivatives, such as betulonic acid, have been found to be cytotoxic, interfering with the proliferation of cells (Hashimoto et al., Bioorg. Med. Chem. 5:2133-2143 (1997)). In addition, no current anti-HIV agent, with the exception of α-interferon, has any effect on release of virus from a chronically infected cell. Thus, the search for new anti-HIV compounds remains timely and important.

The present invention is directed to overcoming these and other deficiencies in the art.

### SUMMARY OF THE INVENTION

One aspect of the present invention relates to a method of inhibiting HIV-1 activity in a cell. This method involves providing a cell infected with HIV-1 and contacting the cell with a compound of Formula I 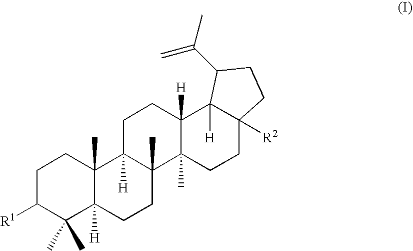
where
R¹ is —CH₃, ═O, —OH, —OCH₃, or —OC(O)CH₃, and
R² is —H, CH₃, —CHO, —CH₂OH, —CH₂OCH₃, —CH₂OC(O)CH₃, —COCH₃, or —COOH,
   or a pharmaceutically acceptable salt or derivative thereof, under conditions effective to inhibit HIV-1 activity in the cell.

Another aspect of the present invention relates to a method of treating HIV-1 infection in a subject. This method involves administering to a subject with HIV-1 infection a therapeutically effective amount of a compound of Formula I where
R¹ is selected from the group consisting of —CH₃, ═O, —OH, —OCH₃, or —OC(O)CH₃, and
R² is selected from the group consisting of —H, —CH₃, —CHO, —CH₂OH, —CH₂OCH₃, —CH₂OC(O)CH₃, —COCH₃, or —COOH,
   or a pharmaceutically acceptable salt or derivative thereof, under conditions effective to treat the subject for HIV-1 infection.

While the prior art discloses a number of different betulinol derivatives having antiviral activity, the betulinol derivatives of the present invention are particularly effective against Human Immunodeficiency Virus and, in particular, HIV-1. Moreover, the betulinol derivatives of the present invention produce anti-HIV-1 activity superior to anti-HIV-1 activity known in the art for other betulinol derivatives. In addition, the compounds of the present invention provide this superior anti-HIV-1 activity without affecting the proliferation of cells.

### BRIEF DESCRIPTION OF THE DRAWINGS

FIG. 1 is a graphic representation of the HIV inhibitory effect of various betulinol derivatives.

FIG. 2 is a graphic representation of % inhibition of HIV infection by certain betulinol derivatives.

FIG. 3 is a graphic representation of % inhibition of HIV infection by varying doses of certain betulinol derivatives.

FIG. 4 is a graphic representation of % inhibition of HIV infection by varying doses of betulinol.

FIG. 5 is a graphic representation of HIV inhibitory effect of varying doses of 28-acetoxy betulin.

FIG. 6 is a graphic representation of % inhibition of AZT versus betulonic acid of H9 cells infected with HIV-IIIB.

FIG. 7 is a graphical representation of cell viability of H9 (lymphoma) cells in the presence of AZT versus betulonic acid.

FIG. 8 is a graphical representation of the anti-HIV activity of betulinol derivatives in CEM (CD4+T) cells.

### DETAILED DESCRIPTION OF THE INVENTION

The present invention relates to a method of inhibiting HIV-1 activity in a cell. This method involves providing a cell infected with HIV-1 and contacting the cell with a compound of Formula I 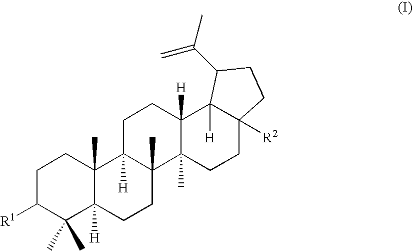
where
R¹ is selected from the group consisting of —CH₃, ═O, —OH, —OCH₃, or —OC(O)CH₃, and
R² is selected from the group consisting of —H, —CH₃, —CHO, —CH₂OH, —CH₂OCH₃, —CH₂OC(O)CH₃, —COCH₃, or —COOH,
   or a pharmaceutically acceptable salt or derivative thereof, under conditions effective to inhibit HIV-1 activity in the cell.

According to the present invention, the compound of Formula I may, for example, have the configurations of R₁ and R₂ as shown in Table 1.

TABLE 1

| R¹ | R² |
|---|---|
| —OCH₃ | —CH₂OCH₃ |
| —OC(O)CH₃(“OAc”) | —CH₂OH |
| —OH | —CH₂OC(O)CH₃ |
| —OH | —COOH |
| —OH | —H |
| ═O | —COOH |
| —OH | —CHO |
| ═O | —CHO |
| —CH₃ | —CH₃ |
| —OAc | —Ac |
| —OH | —Ac |
| —OAc | —H |

In a preferred embodiment, the compound of Formula I is betulin dimethyl ether, of the formula: 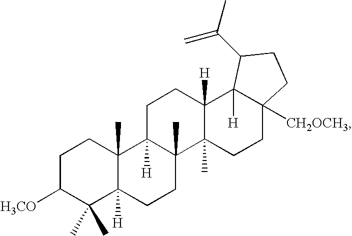
3-acetoxy betulin, of the formula: 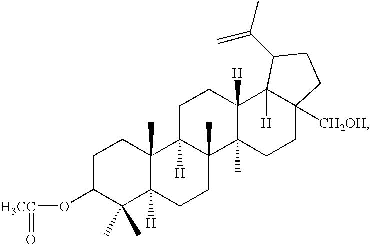
28-acetoxy betulin, of the formula: 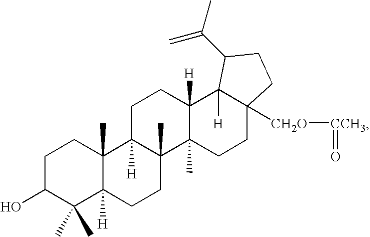
or pharmaceutically acceptable salts or derivatives thereof.

In another preferred embodiment, a combination of compounds of Formula I are employed in the methods of the present invention, provided the combination has at least one compound of Formula I which is betulin dimethyl ether, 3-acetoxy betulin, 28-acetoxy betulin, or pharmaceutically acceptable salts and derivatives thereof.

Compounds of Formula I are synthesized by standard methods that are well known in the art. For example, detailed instructions on how to synthesize and prepare compounds of Formula I are set forth in U.S. Pat. No. 6,890,533, to Bomshteyn et al., which is hereby incorporated by reference in its entirety.

Immunoconjugates of the compounds of Formula I are also suitable in carrying out the methods of the present invention. In one embodiment, immunoconjugates are prepared by attaching an antibody directly to either R¹ or R² of the compound of Formula I. Alternatively, antibodies may be attached to a compound of Formula I via a spacer molecule. A detailed description of methods of attaching antibodies to betulin and betulin-related compounds, as well as preferred immunoconjugates for carrying out the methods of the present invention, are set forth in U.S. Pat. No. 6,890,533, to Bomshteyn et al., which is hereby incorporated by reference in its entirety. A preferred type of antibody for use in the invention is an immunoglobulin which is a gammaglobulin. IgG, IgA, IgE, and IgM subclasses are particularly preferred. Some representative immunoglobulins are monoclonal or polyclonal antibodies to human or animal tumor associated antigens; human B- and T-cell antigens; human Ia antigens; viral, fungal and bacterial antigens; and cells involved in human inflammatory or allergic reactions.

Methods for preparing antibodies and monoclonal antibodies to particular haptenic or antigenic target substrates are described in Goding, Monoclonal Antibodies: Principles and Practice, 2nd. ed., New York:Academic Press, (1986); Kennett et al., Monoclonal Antibodies, New York: Plenum Press (1980); U.S. Pat. No. 4,423,147 to Secher et al.; U.S. Pat. No. 4,381,292 to Bieber et al.; U.S. Pat. No. 4,363,799 to Kung et al.; U.S. Pat. No. 4,350,683 to Galfre et al.; U.S. Pat. No. 4,127,124 to Clagett et al., which are hereby incorporated by reference.

The step of “contacting a cell” with compounds of Formula I can be carried out as desired, including, but not limited to, contacting cells in culture in a suitable growth medium. Alternatively, mice, rats or other mammals are injected with compounds.

Another aspect of the present invention relates to a method of treating HIV-1 infection in a subject (e.g. a human). This method involves administering to a subject with HIV-1 infection a therapeutically effective amount of a compound of Formula I, where
R¹ is selected from the group consisting of —CH₃, ═O, —OH, —OCH₃, or —OC(O)CH₃, and
R² is selected from the group consisting of —H, —CH₃, —CHO, —CH₂OH, —CH₂OCH₃, —CH₂OC(O)CH₃, —COCH₃, or —COOH,
   or a pharmaceutically acceptable salt or derivative thereof, under conditions effective to treat the subject for HIV-1 infection.

In carrying out the method of treating HIV-1 infection in a subject, a therapeutically effective amount of a compound of Formula I is preferably administered to the subject to treat the subject for AIDS. Alternatively, the administering step is carried out to prevent AIDS in the subject infected with HIV-1.

As used here, the term “treating” means amelioration, prevention or relief from the symptoms and/or effects associated with HIV-1 infection, and includes the prophylactic administration of a compound of Formula I, or a pharmaceutically acceptable salt or derivative thereof, to substantially diminish the likelihood or seriousness of the condition.

The relative activity, potency, and specificity of the compound of Formula I may be determined by a pharmacological study in animals, for example, according to the method of Nyberg et al., Psychopharmacology 119:345-348 (1995), which is hereby incorporated by reference in its entirety. Although the differential metabolism among patient populations can be determined by a clinical study in humans, less expensive and time-consuming substitutes are provided by the methods of Kerr et al., Biochem. Pharmacol. 47:1969-1979 (1994), which is hereby incorporated by reference in its entirety and Karam et al., Drub Metab. Discov. 24:1081-1087 (1996), which is hereby incorporated by reference in its entirety. The potential for drug-drug interactions may be assessed clinically according to the methods of Leach et al., Epilepsia 37:1100-1106 (1996), which is hereby incorporated by reference in its entirety, or in vitro according to the methods of Kerr et al., Biochem. Pharmacol. 47:1969-1979 (1994), which is hereby incorporated by reference in its entirety and Turner et al., Can. J. Physio. Pharmacol. 67:582-586 (1989), which is hereby incorporated by reference in its entirety.

The magnitude of a prophylactic or therapeutic dose of the compound of Formula I, or a pharmaceutically acceptable salt or derivative thereof, will vary with the nature and severity of the condition to be treated and the route of administration. The dose, and perhaps the dose frequency, will also vary according to the age, body weight, and response of the individual subject. The total daily dose of compounds of Formula I, or pharmaceutically acceptable salts or derivatives thereof, may be administered in single or divided doses.

It is further recommended that children, subjects over 65 years old, and those with impaired renal or hepatic function, initially receive low doses and that the dosage be titrated based on individual responses and blood levels. It may be necessary to use dosages outside these ranges in some cases, as will be apparent to those of ordinary skill in the art. Further, it is noted that the clinician or treating physician knows how and when to interrupt, adjust, or terminate therapy in conjunction with and individual subject's response.

Any suitable route of administration may be employed. For example, oral, rectal, intranasal, parenteral, subcutaneous, intramuscular, intravaginally, intravenous, intraperitoneal, intracavitary or intravesical instillation, intraocular, intraarterial, and intralesional routes may be used, as well as application to mucous membranes, such as, that of the nose, throat, and bronchial tubes. Dosage forms include, for example, tablets, troches, dispersions, suspensions, solutions, capsules, powders, solutions, suspensions, emulsions, and patches.

The compound of Formula I may, for example, be incorporated into a biocompatible matrix and delivered intravaginally. As a prophylactic delivery system, the compound of Formula I may, for example, be incorporated within, or coated on, a condom.

Pharmaceutical compositions of the present invention include at least one compound of Formula I, a pharmaceutically acceptable salt or derivative thereof, or combinations thereof. Such compositions may include a pharmaceutically acceptable carrier, and optionally, other therapeutic ingredients or excipients.

The term “pharmaceutically acceptable salt thereof” refers to salts prepared from pharmaceutically acceptable, non-toxic acids including inorganic acids and organic acids, such as, for example, acetic acid, benzenesulfonic (besylate) acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluenesulfonic acid.

The pharmaceutical compositions may be conveniently presented in unit dosage form, and may be prepared by any of the methods well known in the art of pharmacy. Preferred unit dosage formulations are those containing an effective dose, or an appropriate fraction thereof, of the active ingredients.

The compositions of the present invention may include a pharmaceutically acceptable carrier. The carrier may take a wide variety of forms, depending on the forms preparation desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by a standard aqueous or nonaqueous technique. Oral and parenteral sustained release dosage forms may also be used.

Oral syrups, as well as other oral liquid formulations, are well known to those skilled in the art, and general methods for preparing them are found in any standard pharmacy school textbook. For example, chapter 86, of the 19th Edition of Remington: The Science and Practice of Pharmacy, entitled “Solutions, Emulsions, Suspensions and Extracts,” describes in complete detail the preparation of syrups (pages 1503-1505, which are hereby incorporated by reference in their entirety) and other oral liquids.

Similarly, sustained release formulations are well known in the art, and Chapter 94 of the same reference, entitled “Sustained-Release Drug Delivery Systems,” describes the more common types of oral and parenteral sustained-release dosage forms (pages 1660-1675, which are hereby incorporated by reference in their entirety). Because they reduce peak plasma concentrations, as compared to conventional oral dosage forms, controlled release dosage forms are particularly useful for providing therapeutic plasma concentrations while avoiding the side effects associated with high peak plasma concentrations that occur with conventional dosage forms.

The solid unit dosage forms can be of the conventional type. The solid form can be a capsule, such as an ordinary gelatin type containing the betulinol derivative and a carrier, for example, lubricants and inert fillers, such as lactose, sucrose, or cornstarch. In another embodiment, these betulinol derivatives can be tableted with conventional tablet bases, such as lactose, sucrose, or cornstarch, in combination with binders, like acacia, cornstarch, or gelatin, disintegrating agents, such as cornstarch, potato starch, or alginic acid, and lubricants, like stearic acid or magnesium stearate.

The pharmaceutical compositions may also be administered in injectable dosages by solution or suspension of these materials in a physiologically acceptable diluent with a pharmaceutical carrier. Such carriers include sterile liquids, such as water and oils, with or without the addition of a surfactants, adjuvants, excipients, or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the pharmaceutical compositions in solution or suspension may be packaged in a pressurized aerosol container together with suitable propellants, for example, hydrocarbon propellants like propane, butane, or isobutane, and with conventional adjuvants. The pharmaceutical compositions may also be administered in a non-pressurized form, such as in a nebulizer or atomizer.

### EXAMPLES

The following examples are provided to illustrate embodiments of the present invention but are by no means intended to limit its scope.

### Example 1

### Preparation of Cell Samples

Human T-B hybridoma cell line 174XCEM was exposed to a low multiplicity of infection (“MOI”) (MOI=1.0) of stock HIV-1 IIIB isolate for 2 hours at 37° C., washed ×3 with phosphate buffered saline (“PBS”), then plated at 250,000 cells/well in the presence of various agents, shown in Table 2. Dimethyl sulfoxide (“DMSO”) buffer was used as a “no virus” control. A commercially available synthetic peptide, thrombospondin peptide (“TSP”), known as having HIV-1 inhibitory activity, was used as an “inhibitory activity” control. Two HIV isolates were used, a patient isolate (“child HIV”), and the standard CXCR4 co-receptor utilizing isolate IIIB.

TABLE 2

| Agent | Concentration |
|---|---|
| TSP peptide (“control”) | 1 μg/ml |
| betulinol (“OL”) | 1 μg/ml (in DMSO) |
| betulonic acid (“BOA”) | 1 μg/ml (in DMSO) |
| 3-acetoxy betulin (“BL”) | 1 μg/ml (in DMSO) |
| betulin dimethyl ether (“BDE”) | 1 μg/ml (in DMSO) |
| 28-acetoxy betulin (“BU”) | 1 μg/ml (in DMSO) |
| betulone aldehyde (“AL”) | 1 μg/ml (in DMSO) |
| betulin diacetate (“BA”) | 1 μg/ml (in DMSO) |

The chemical structures of betulin dimethyl ether, 3-acetoxy betulin, and 28-acetoxy betulin have been previously disclosed herein. The chemical structures of betulonic acid, betulone aldehyde, and betulin diacetate are:

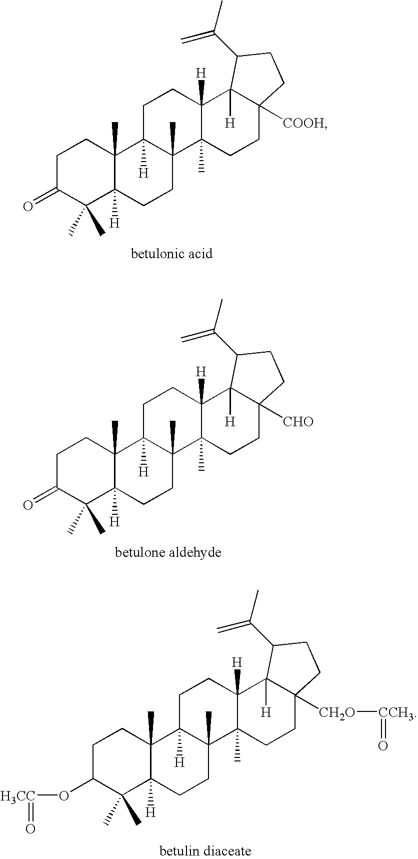

### Example 2

### Assay for HIV-1 Inhibitory Effect

The assay methods described herein are known in the art, and are described in detail, for example, in Crombie et al., J. Exp. Med. 187:25-35 (1998), which is hereby incorporated by reference in its entirety.

Cultures were maintained in culture medium (RPMI-1640+10% fetal bovine serum (“FBS”)) for 4 days, the culture supernatants were then collected, lysed with Triton®-X 100 surfactant, and HIV-1 gag (p24) antigen activity assessed by a standard technique, the Antigen Capture ELISA (enzyme-linked immunosorbent assay) (Roche-NEN).

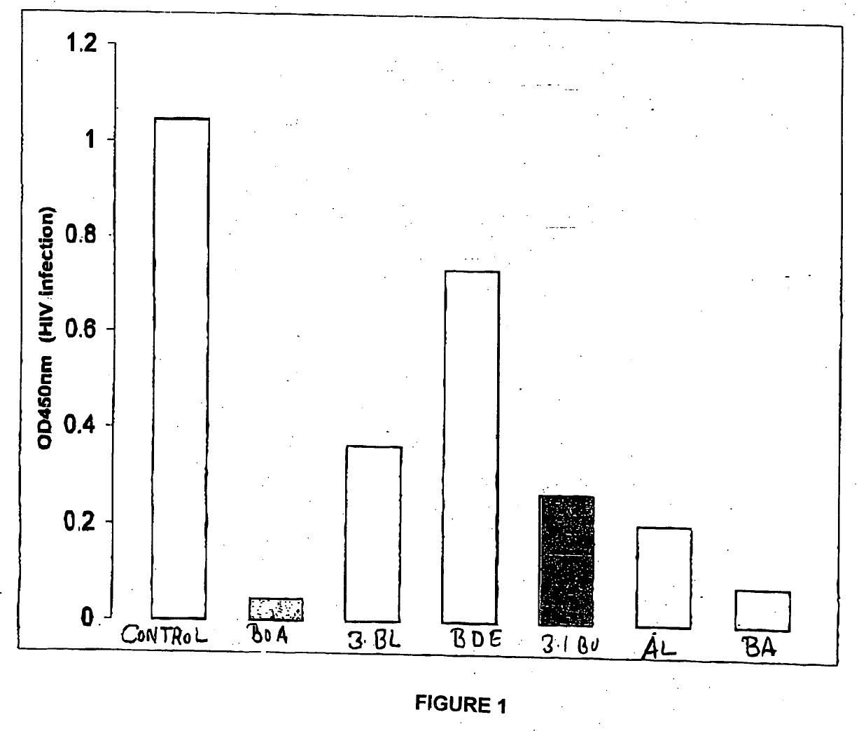

Results are shown in FIG. 1. Data are presented in optical density (“OD”) units, which are linear with ng/ml of p24 Ag from 0.15 to 1.5 OD, and can be converted to pg/ml of HIV-1 antigen using a standard curve. (Note that the “no virus” DMSO control had an OD reading<0.05, and is not shown in FIG. 1; “control” represents the “inhibitory effect” control, TSP peptide.)

Surprisingly, as is clearly seen from FIG. 1, betulin dimethyl ether (BDE), 3-acetoxy betulin (BL) and 28-acetoxy betulin (BU), provide anti-HIV-1 activity superior to that previously disclosed in the art for other betulinol derivatives. The anti-HIV activity of betulonic acid and betulin diacetate has previously been disclosed, for example, in U.S. Pat. No. 6,172,110 to Lee et al., which is hereby incorporated by reference in its entirety. The anti-HIV activity of betulone aldehyde has previously been disclosed, for example, in U.S. Pat. Nos. 5,869,535 and 6,225,353 to Pezzuto et al., which are hereby incorporated by reference in their entirety.

### Example 3

### Effect on Cell Viability

The cell samples were assessed by trypan blue dye exclusion at four days and seven days. Unlike prior art betulin derivatives, such as, for example, betulonic acid, betulin dimethyl ether, 3-acetoxy betulin, and 28-acetoxy betulin had no effect on total cell number or cell viability.

### Example 4

### Anti-HIV-1 Effect

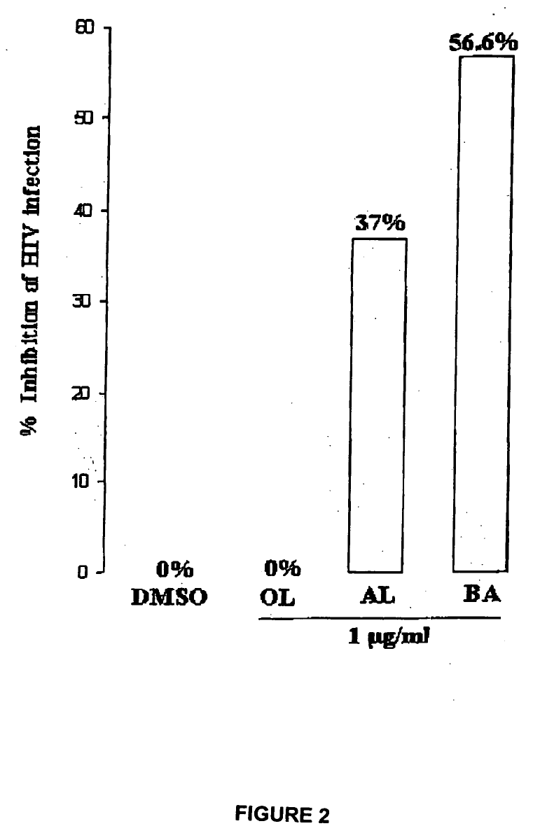

The known anti-HIV-1 inhibitory peptide thrombospondin (TSP), produced 92% inhibition. The DMSO control and OL showed no effect. As shown in FIG. 2, betulone aldehyde (AL) showed 37% inhibition and betulin diacetate (BA) showed 57% inhibition.

### Example 5

### Dose Dependent Anti-HIV-1 Effect

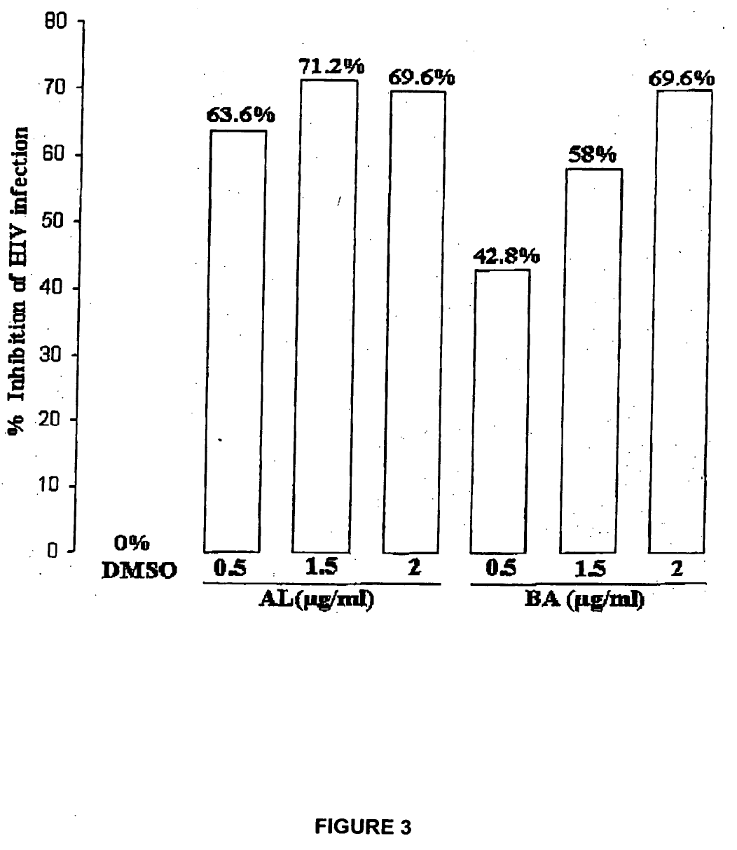

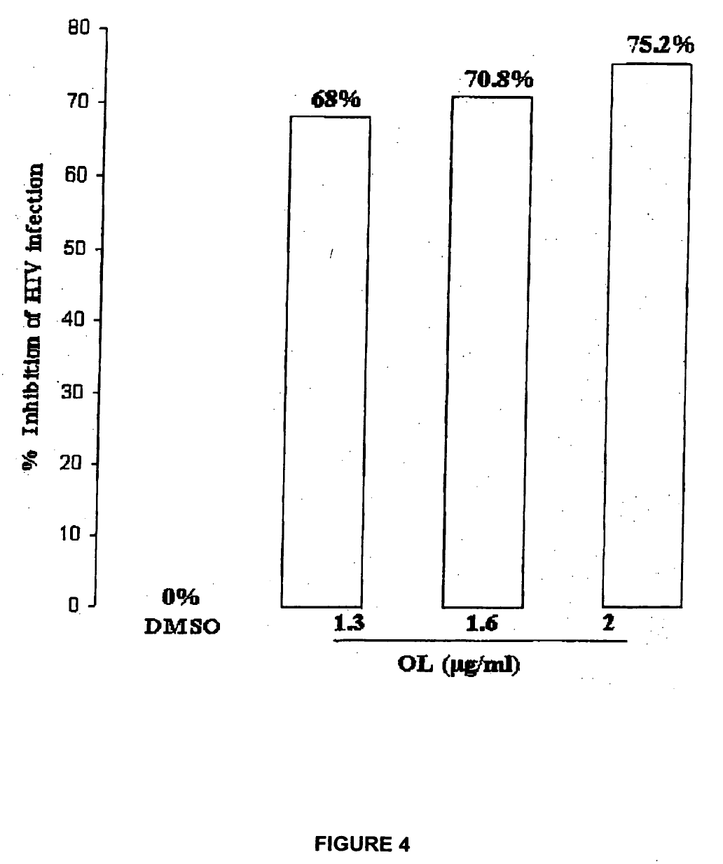

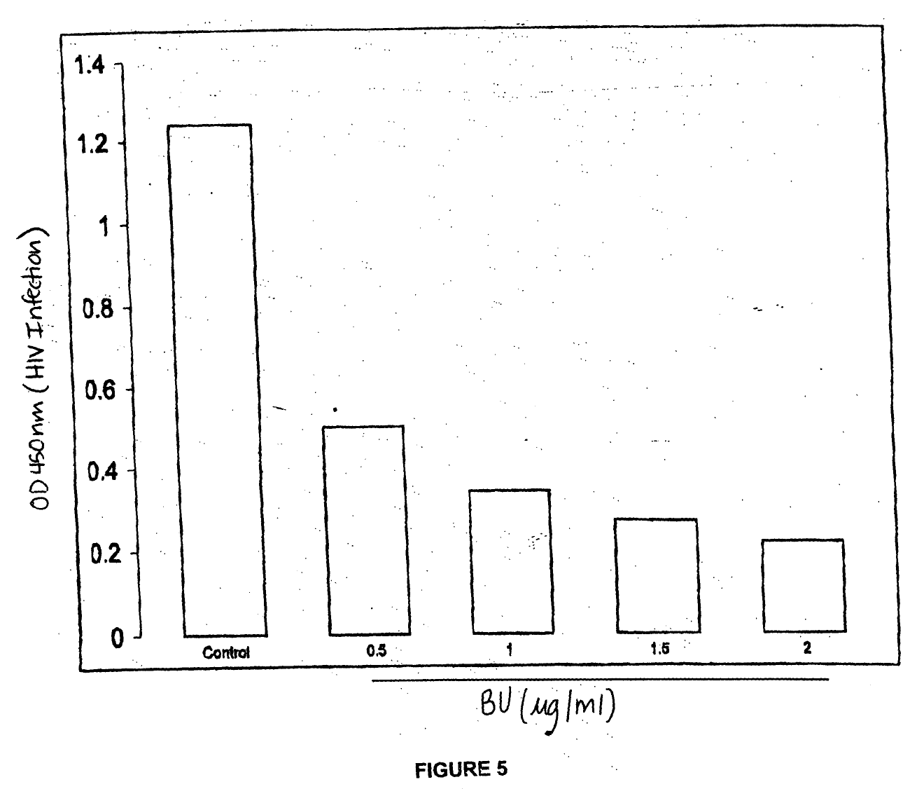

As illustrated in FIG. 3, betulone aldehyde (AL) and betulin diacetate (BA) were tested for dose-related effects, with doses of 0.5, 1.5, and 2 μg/ml. Progressive increases in anti-HIV effect were shown, again without cell toxicity. As illustrated in FIG. 4, varying doses of the parental compound betulinol (OL) (1.3, 1.6, and 2 μg/ml) showed increasing anti-HIV effect. As illustrated in FIG. 5, varying doses of 28-acetoxy betulin (BU) (0.5, 1, 1.5, and 2 μg/ml) showed comparable anti-HIV-1 activity. Doses higher than 2 μg/ml could not be used, because the concentration of the vehicle used to dissolve these agents (DMSO) would be too high for the present culture system.

### Example 6

### Effect on 174XCEM Cells Chronically Infected with Another HIV-1 Isolate

Agents were also evaluated for an effect on 174XCEM cells chronically infected with another HIV-1 isolate. In this system, the standard anti-HIV TSP peptide had no effect. Betulinol and betulone aldehyde had minimal effect. Betulin diacetate showed 20% inhibition at the single dose tested at 1 μg/ml. Although the degree of activity seen in this experiment with chronically infected cells is modest, it should be noted that no current anti-HIV agent, with the exception of α-inteferon, has any effect on release of virus from a chronically infected cell.

### Example 7

### Comparison of Anti-HIV-1 Activity of Betulinol Derivatives with Known HIV Inhibitors

Viral isolates: standard HIV-1 lab isolate IIIB, highly sensitive to all known anti-HIV compounds, and two patient isolates obtained from Haiti, with varying degrees of anti-HIV drug sensitivity.

Target cells: CD4+ Jurkat and CEM-SS human T lymphoblasts, were grown in culture medium (RPMI 1640 plus 10% heat-inactivated FBS). Human peripheral blood mononuclear cells (“PBMC”) were derived from heparinized venous blood by density gradient centrifugation using Ficol-paque (Amersham-Pharmacia). For HIV infections, PBMCs were pre-activated with 1 μg/ml phytohemagglutinin (“PHA”) and 32 U/ml interleukin-2 (“IL-2”) for 2-3 days prior to exposure to HIV-1.

HIV infection: HIV-1 infections were performed as previously described herein. Briefly, 2.5×105 target cells (cell lines or PHA-activated PBMCs) were exposed to stock virus (500 pg of HIV-1 p24 antigen) for 2 h at 37° C., washed twice with PBS, and replated with fresh medium. One half of the culture supernatants were removed from each well every 3-4 days and replaced with fresh medium. At various times after viral inoculation, HIV-1 activity was determined by antigen capture ELISA (Roche-NEN) for HIV-1 p24 gag protein in Triton®-X 100 solubilized culture supernatants, as described.

Drugs: The reverse transcriptase inhibitor AZT and the HIV protease inhibitors ritonavir and nelfinavir were used alone, and in potential synergy experiments with compounds of Formula I. The drugs were added to target cell cultures either before or after the two hour incubation of target cells with virus. AZT was used in concentrations of 0.01-5 μM and the protease inhibitors at concentrations of 0.5-10 μM.

### Example 8

### Effect of Betulinol Derivatives on HIV-1 RT and Protease, Using Purified Viral Enzymes

To evaluate the mechanism of action of compounds of Formula I, direct effects on the two key viral enzymes were measured.

Purified viral enzymes: Reverse transcriptase corresponding to native RT dimer (66 kd/51 kd) purity>98% was obtained from the National Institute of Health (“NIH”) AIDS Research and Reference Reagent Program (catalog no. 3555). HIV-1 protease (KIIA, molecular weight 10.7 kd) was obtained from the same source (catalog no. 4375). The protease is identical to wild-type HIV-1 IIIB (HXB2 clone) protease, except for four amino acid substitutions which render it highly resistant to autoproteolysis and oxidative inactivation, making in vitro assays easier.

HIV enzyme assays: HIV RT was assessed by ELISA (Roche-NEN) using the purified enzyme with polyrA/T as substrate and AZT as a positive control, with varying concentrations of compounds of Formula I added. HIV protease was similarly assessed using, as substrate, a 9 amino acid synthetic peptide spanning the p17/p24 junction of HIV gag. Specific activity against this peptide is 12.1 μM/min/mg over 10 min.

### Example 9

### Effect of Betulinol Derivatives on Cell Proliferation

Compounds of Formula I were evaluated for cellular effects which might indicate toxicity or non-specific anti-viral properties. Effects of varying doses of compounds of Formula I on T cell proliferation was assessed by standard methods. In addition, potential induction of apoptosis by these compounds at the anti-HIV doses used, as well as at high concentrations of compounds was assessed.

Apoptosis identification: Levels of apoptosis were assessed by TO-PRO-3 staining (VanHooijdonk, et al., Cytometry 17:185-189 (1994), which is hereby incorporated by reference in its entirety). Briefly, cells were air dried on slides fixed in 4% paraformalydehyde for 10 min. at room temperature, washed with PBS, and treated with 70% EtOH for 15 min. at −20° C. The slides were fixed in a 1:9 solution of acetic acid:ethanol for 1 h, washed, then treated with 2% Triton®X-100 for 2 min., followed by exposure to RNAse A for 20 min. at 4° C. 2-3 drops of a 0.5 μM solution of TO-PRO-3 (Molecular Probes, Invitrogen Life Technologies, Eugene, Oreg.) were added and slides incubated for 10 min. at room temperature in the dark. Slides were then washed, treated with the anti-quenching agent Vectashield (Vector Labs, Inc., Burlingame, Calif.), sealed, and visualized with a fluorescent microscope for evidence of membrane and nuclear integrity.

### Example 10

### Effect of Betulinol Derivatives on HIV Binding to Target Cells

This is a further investigation of the mechanism of action of compounds of Formula I. It assessed whether these compounds have any membrane-specific properties, interfering with HIV gp120 envelope binding to the two receptors for the virus, CD4 and co-receptor (CXCR4 or CCRS).

HIV envelope proteins: Recombinant HIV-1 gp120 of CXCR4 phenotype (obtained from NIH AIDS Program, described above) and CCR5 phenotype were used.

Cell targets: T cell targets bearing HIV co-receptors and CD4 (CEM-T) or co-receptors but no CD4 (CEM-SS) were utilized. Different target cells bearing CXCR4 but not CCR5 (M07E) were also used.

Cell surface SDF-1/gp120 binding assays: Binding of HIV envelope to CXCR4 and its competition with SDF-1 was assessed by a very sensitive fluorescence binding assay. This involved oligomeric X4 gp160, representing multimers of gp120 and its non-covalently bound transmembrane portion, gp41. This type of assay is necessitated by the low affinity of the gp120-CXCR4 interaction in vitro, as contrasted with gp120 binding to its alternate chemokine receptor CCR5 (Lin et al., J. Virol. 77:931-942 (2003), which is hereby incorporated by reference in its entirety). Detailed methods, including demonstration of specificity and CD4 independence of the binding assay, have been published (Staudinger et al., Biochem. Biophys. Res. Comm. 280:1003-1007 (2001); Bandres et al., J. Virol, 72:2500-2504 (1998), which are hereby incorporated by reference in their entirety).

Varying concentrations of oligomeric X4 gp160 were added for 1 h at 37° C. to target cells. The cells were then washed and incubated with 10 μg/ml of human mAb 1331A, specific for the C terminus of gp120, or with a human mAb against the HIV-1 core protein p24 as a control, both conjugated to phycoerythrin (“PE”), and fluorescence intensity assessed. Displacement of a fixed amount of oligomeric viral envelope, as detected by the human anti-gp120 mAb, by increasing amounts of compounds of Formula I were examined. Positive controls for CD4 (monoclonal antibody) CXCR4 (SDF-1,500 to 1500 ng/ml), and CCP5 (1500 ng/ml RANTES) were included.

### Example 11

### Effect of Betulinol Derivatives on HIV Promoter (LTR)-Driven Transcription

The effects of compounds of Formula I on HIV promoter (LTR)-driven transcription, emphasizing HIV-1 Tat and NPκB activity was evaluated.

Plasmid constructs, plasmid transfections and reporter assays: The reporter plasmid pC15CAT (Arya et al., Science 229:69-73 (1985), which is hereby incorporated by reference in its entirety) contains sequences for SV40 regulatory genes, bacterial chloramphenical acetyl transferase (“CAT”), and the HIV-1 long terminal repeat (“LTR”). The HIV-1 tat plasmid pCV-1 (Arya et al., Science 229:69-73 (1985), which is hereby incorporated by reference in its entirety) contains a 1.8 kb cDNA fragment encompassing both exons of tat. For transfections, cells were washed with serum-free RPMI-1640, and 2×10⁶ cells per condition are resuspended in 1 ml of Optimum media (Gibco, Life Technologies, Gaithersburg, Md.) along with 2-6 μg plasmid DNA and DMRIE-C transfection reagent (Gibco, Life Technologies, Gaithersburg, Md.). Cells were incubated at 37° C. for 5 h, and fresh RPMI 1640 containing 10% FBS added. 36 h after transfection, select samples were treated with compound. CAT assays were performed using a kit (Roche), as per the manufacturer's directions.

Electrophoretic Mobility Shift Assay (“EMSA”): This is a standard assay for assessing NFκ activity. Target cells were exposed to compounds of Formula I alone, in the presence of a known NFκB activator (TNF-α), or with HIV-1 for 48 h. Nuclear extracts were then prepared using a Nuclear extract kit (Sigma). 10 μg of nuclear extract was dissolved in a buffer containing 1 ng of 32P-5′ end labeled, κB probe, 1 μg of poly(dI-dC), 50 ng of sonicated salmon sperm DNA, 10 mM MgCl₂, 25 mM KCl, 1 mM DTT, 12.5 mM HEPES pH 7.8, 10% glycerol, and 0.05% Nonidet p-40. Mixtures were incubated for 15 min. at 4° C. and protein bound DNA complexes were analyzed by electrophoresis on a 6% polyacrylamide gel. Controls include a competition assay with unlabelled κB oligonucleotide added at a 50 fold excess to probe.

### Example 12

### Synthesis and Characterization of Betulonic Acid

A batch of 500 mg of betulinol was added to a suspension of freshly activated 1.2 g celite, 1.2 g florisil, 500 mg sodium acetate, and 1.2 g pyridinium chlorochromate in 25 mL of CH₂Cl₂. The mixture was stirred for 2 hrs, and then filtered through a column of mesh and 60 Angstrom silica gel 230-400 (Merck & Co., Inc., Whitehouse Station, N.J.). The filtrate was evaporated in vacuum. The residue was subjected to column chromatography to recover 370 mg betulone aldehyde as white solid. The betulone aldehyde was dissolved in a mixture of 877 mg NaH₂PO₄.H₂O and 17 mL CH₃CN—H₂O and cooled to 0-5° C. 220 μL of thirty percent of aqueous H₂O₂ and 200 mg of NaClO₂ dissoloved in 16 mL water were added in tandem. The mixture was brought to room temperature and stirred for one hour. The reaction was quenched by the addition of 380 mg Na₂S₂O₅ and extracted in ethyl acetate. The organic extract was washed with water and brine, dried by (Na₂SO₄), filtered, and concentrated. The residue was subjected to column chromatography to recover 550 mg betulonic acid as white solid powder. The synthetic scheme is illustrated as follows:

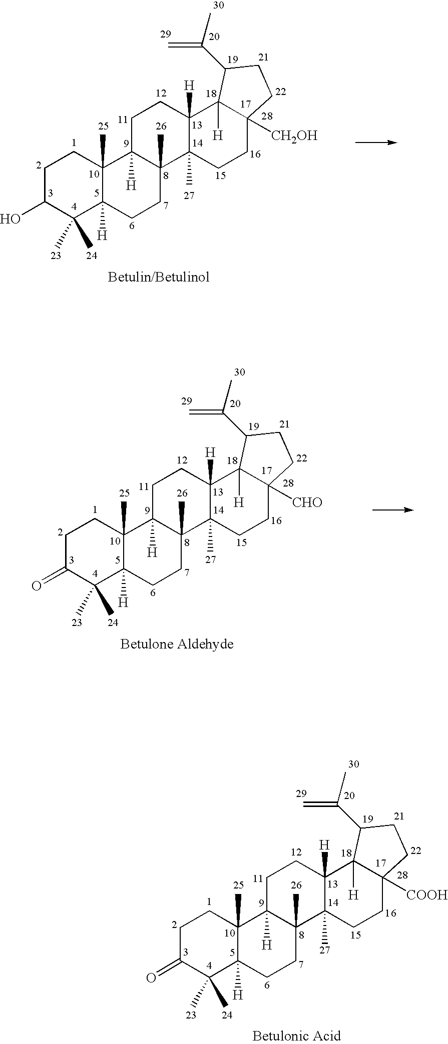

### Example 13

### Determination of the Inhibition of HIV Infection by Betulinol Derivatives Using H9 (Lymphoma Cells)

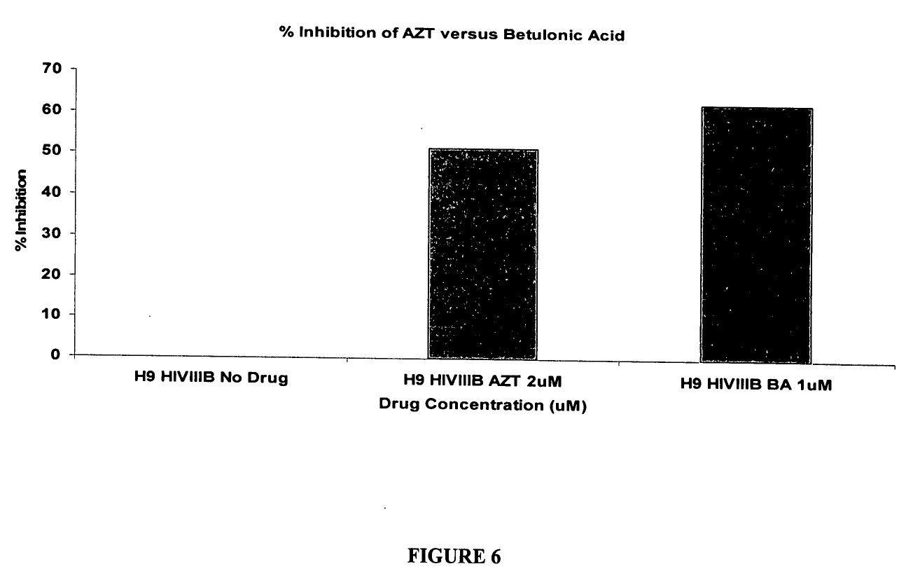

1.5×10⁵ of H9 cells were exposed to a stock HIV-1 IIIB late (at an MOI of 1.0) at 37° C. for 2 hours, washed 3 times with PBS, and plated out in 1 mL of RPMI media containing 10% FBS in the presence of betulonic acid with or without AZT. On day 3, half of the media (0.5 mL) was replaced with fresh media and appropriate drugs. On day 7, the culture supernatants were collected, solubilized in Triton-X100, and HIV-1 Gag antigen p24 were assessed and presented in optical density (“OD”) units using a standard assay (p24 ELISA Kit from Perkin Elmer, Wellesley, Mass.). Results are set forth in FIG. 6. The decrease of OD units represented the drug inhibition effects on HIV infection. This method is from Crombie et al., J. Exp. Med. 187:25-35 (1998), which is hereby incorporated by reference in its entirety.

### Example 14

### Viability of Lymphoma Cells in the Presence of AZT Versus Betulonic Acid

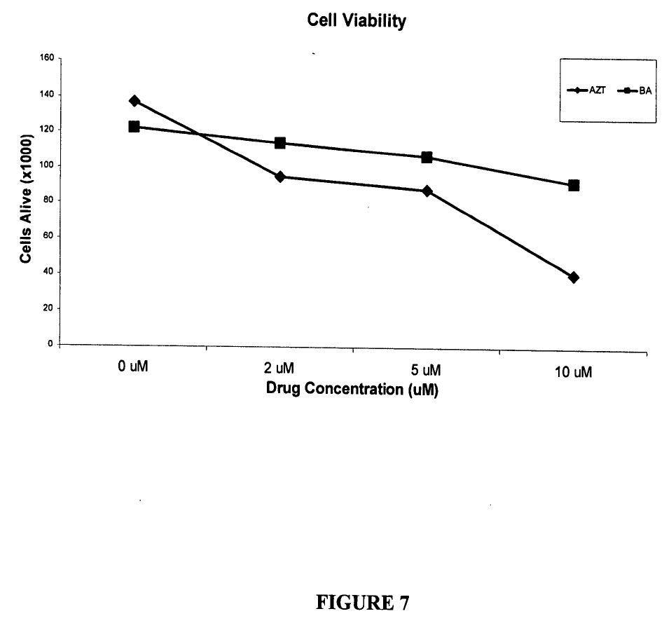

1.5×10⁵ of H9 (lymphoma) cells were plated in each culture well in 1 mL of RPMI media containing 10% FBS in the presence of 0, 2, 5, 10, and 20 mM of betulonic acid and AZT and incubated at 37° C. On day 3, the drug effects on cell viability were assessed using Trypan Blue Dye Exclusion Assay. Results are set forth in FIG. 7. The data is presented as both living cell counts and percentage. Chemical resources were obtained through Sigma Aldrich.

### Example 15

### Determination of the Inhibition of HIV Infection by Betulinol Derivatives Using Crombie's Method

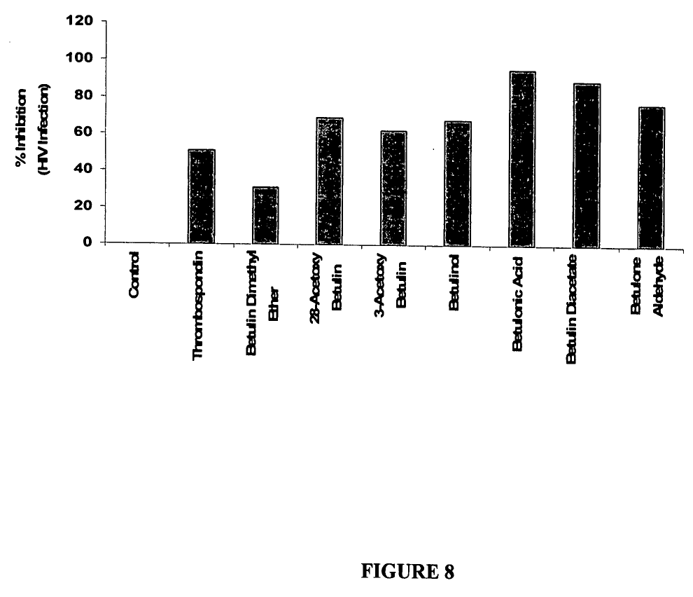

Acute HIV infection was performed using HIV-1 isolate IIIB stock virus. In brief, CEM (CD4+T) cells (2.5×10⁵ target cells) were exposed to stock virus at a MOI of either 0.02 or 0.15 for 2 h at 37° C., washed twice with PBS, and replated in tissue culture microwells with 0.3 ml of fresh culture medium. Compounds of Formula I dissolved in DMSO were added into the culture and were tested for anti-HIV activity with reference to thrombospondin (TSP), a known anti-HIV drug. Three days after inoculation, one half of culture supernatant from each well was replaced with fresh medium. HIV activity was determined on day seven using an ELISA antigen capture assay for HIV-1 p24 (Gag) core protein (Dupont Medical Products, Boston, Mass.) with Triton X-100 solubilized culture supernatants. Inhibition was calculated as percent of the control. Thrombospondin (TSP) was used at a concentration of 1 mg/mL and yielded an inhibition of 51%. Compounds of Formula I were also used at a concentration of 1 ug/mL. Results are set forth in FIG. 8.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of inhibiting HIV-1 activity in a cell, said method comprising:
providing a cell infected with HIV-1 and contacting the cell with a compound of Formula I 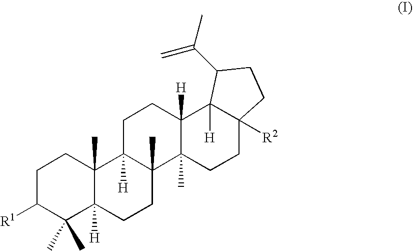
wherein
R¹ is selected from the group consisting of —CH₃, ═O, —OH, —OCH₃, and —OC(O)CH₃; and
R² is selected from the group consisting of —H, —CH₃, —CHO, —CH₂OH, —CH₂OCH₃, —CH₂OC(O)CH₃, —COCH₃, and —COOH,
or a pharmaceutically acceptable salt or derivative thereof, under conditions effective to inhibit HIV-1 activity in the cell.

2. The method according to claim 1, wherein the compound of Formula I is selected from the group consisting of betulin dimethyl ether, 3-acetoxy betulin, 28-acetoxy betulin, and pharmaceutically acceptable salts and derivatives thereof.

3. The method according to claim 1, wherein the cell is contacted with a combination of compounds of Formula I, wherein the combination comprises at least one compound of Formula I selected from the group consisting of betulin dimethyl ether, 3-acetoxy betulin, 28-acetoxy betulin, and pharmaceutically acceptable salts and derivatives thereof.

4. The method according to claim 1, wherein the compound of Formula I is betulin dimethyl ether or a pharmaceutically acceptable salt or derivative thereof.

5. The method according to claim 1, wherein the compound of Formula I is 3-acetoxy betulin or a pharmaceutically acceptable salt or derivative thereof.

6. The method according to claim 1, wherein the compound of Formula I is 28-acetoxy betulin or a pharmaceutically acceptable salt or derivative thereof.

7. A method of treating HIV-1 infection in a subject, said method comprising:
administering to a subject with HIV-1 infection a therapeutically effective amount of a compound of Formula I 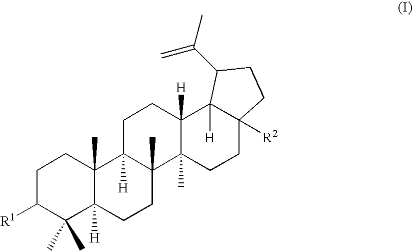
wherein
R¹ is selected from the group consisting of —CH₃, ═O, —OH, —OCH₃, and —OC(O)CH₃; and
R² is selected from the group consisting of —H, —CH₃, —CHO, —CH₂OH, —CH₂OCH₃, —CH₂OC(O)CH₃, —COCH₃, and —COOH,
or a pharmaceutically acceptable salt or derivative thereof, under conditions effective to treat the subject for HIV-1 infection.

8. The method according to claim 7, wherein the compound of Formula I is selected from the group consisting of betulin dimethyl ether, 3-acetoxy betulin, 28-acetoxy betulin, and pharmaceutically acceptable salts and derivatives thereof.

9. The method according to claim 7, wherein a combination of compounds of Formula I are administered, wherein the combination comprises at least one compound of Formula I selected from the group consisting of betulin dimethyl ether, 3-acetoxy betulin, 28-acetoxy betulin, and pharmaceutically acceptable salts and derivatives thereof.

10. The method according to claim 7, wherein the compound of Formula I is betulin dimethyl ether or a pharmaceutically acceptable salt or derivative thereof.

11. The method according to claim 7, wherein the compound of Formula I is 3-acetoxy betulin or a pharmaceutically acceptable salt or derivative thereof.

12. The method according to claim 7, wherein the compound of Formula I is 28-acetoxy betulin or a pharmaceutically acceptable salt or derivative thereof.

13. The method according to claim 7, wherein said administering is carried out to treat the subject for AIDS.

14. The method according to claim 7, wherein said administering is carried out to prevent AIDS in the subject infected with HIV-1.
